(19) United States
(12) Patent Application Publication
(10) Pub. No.: US 2024/0261042 A1
(43) Pub. Date: Aug. 8, 2024

(54) DRAPE UNIT WITH COVER, LOCKING COVER, AND MEDICAL ROBOT

(71) Applicant: Riverfield Inc., Tokyo (JP)

(72) Inventor: Naoya MORITA, Tokyo (JP)

(73) Assignee: Riverfield Inc., Tokyo (JP)

(21) Appl. No.: 18/600,386

(22) Filed: Mar. 8, 2024

Related U.S. Application Data
(63) Continuation of application No. PCT/JP2021/037588, filed on Oct. 11, 2021.

(51) Int. Cl. A61B 34/30; A61B 46/10

(52) U.S. Cl. CPC A61B 34/30; A61B 46/10

(57) ABSTRACT

A drape unit disposed between a medical robot that holds a surgical instrument and the surgical instrument to isolate the surgical instrument and the medical robot from each other, the drape unit including a drape unit and a locking cover. The draft unit comprising a slider having a first engagement portion and a second engagement portion, and a separator main body having a through-hole, and the locking cover, configured to be detached and reattached to the drape unit, comprising: a main body portion configured to cover an entirety of the through-hole, a holding portion that detachably holds a relative position of the main body portion to the separator main body, and a slider holding portion provided so as to hold a relative position between the main body portion and the slider, the slider holding portion being configured to engage with the second engagement portion.

### CROSS-REFERENCE TO RELATED APPLICATIONS

This application is a continuation of International Application No. PCT/JP2021/037588 filed Oct. 11, 2021, the contents of which being incorporated by reference herein in its entirety.

### BACKGROUND

The present disclosure relates to a drape unit with cover that isolates a medical robot and a surgical instrument from each other and, more particularly to a locking cover and a medical robot.

Surgery using medical robots is attracting attention as a technique that enhances the possibility of not only reducing the burden on a surgeon but also the burden on a patient as well as the possibility of remote medical care through highly accurate and stable treatment. During operation of a medical robot, the surgical instrument is sterilized, but the medical robot side is not as sterilized. For this reason, in surgery utilizing a medical robot, it is advantageous that the medical robot side is covered with a drape in order to isolate the clean area and the unclean area from each other.

However, in order to isolate the clean area and the unclean area from each other, it is necessary to cover an entire drive section, making it difficult to downsize the device. In addition, the drape unit, which is an intermediate member, is interposed between the medical robot and the surgical instrument, but when reattaching the drape unit to the medical robot, if it takes time to align the drive section and the movable portion of the drape unit, it may result in incorrect reattachment or it may take time for the reattachment.

### SUMMARY

It is an aspect to provide a drape unit with cover to allow the clean area and unclean area to be sufficiently isolated from each other and enable the reattachment of the drape unit interposed between the medical robot and the surgical instrument to be completed accurately and in a short time.

According to an aspect of one or more embodiments, there is provided a drape unit, including: a drape unit disposed between a medical robot that holds a surgical instrument and the surgical instrument to isolate the surgical instrument and the medical robot from each other, the drape unit comprising: a slider having a first engagement portion engaged with a power transmission part of the medical robot configured to transmit power in a forward/backward direction and a second engagement portion engaged with a movable part of the surgical instrument, the slider transmitting power received from the power transmission part to the movable part; and a separator main body having a through-hole through which at least a part of the slider is inserted, the separator main body being detachably attached to the medical robot; and a locking cover configured to be detached and reattached to the drape unit, the locking cover comprising: a main body portion configured to cover an entirety of the through-hole; a holding portion that detachably holds a relative position of the main body portion to the separator main body; and a slider holding portion provided on the main body portion so as to hold a relative position between the main body portion and the slider, the slider holding portion being configured to engage with the second engagement portion.

### BRIEF DESCRIPTION OF DRAWINGS

The above and/or the other aspects will become apparent and more readily appreciated from the following description of exemplary embodiments, taken in conjunction with the accompanying drawings, in which:

FIG. 1 is a perspective view for exemplifying a medical robot and a drape unit according to some embodiments;

FIG. 2 is a perspective view for exemplifying the attaching state of a surgical instrument according to some embodiments;

FIG. 3 is a perspective view for exemplifying attachment of the surgical instrument to the drape unit according to some embodiments;

FIG. 4 is a plan view for exemplifying a driving portion of the medical robot according to some embodiments;

FIG. 5 is an exploded perspective view of the drape unit according to some embodiments;

FIG. 6 is a cross-sectional view for exemplifying the configuration of the drape unit according to some embodiments;

FIG. 7 is a perspective view for exemplifying a drape unit with cover according to some embodiments;

FIG. 8A is an exploded perspective view for exemplifying the drape unit with cover according to some embodiments;

FIG. 8B is an enlarged view of part A of FIG. 8A;

FIG. 9A is a diagram for exemplifying a locking cover according to some embodiments;

FIG. 9B is a diagram for exemplifying the locking cover according to some embodiments;

FIG. 10A is a schematic diagram for exemplifying a method of reattaching the drape unit according to some embodiments;

FIG. 10B is a schematic diagram for exemplifying the method of reattaching the drape unit according to some embodiments;

FIG. 10C is a schematic diagram for exemplifying the method of reattaching the drape unit according to some embodiments;

FIG. 11A is a schematic diagram for exemplifying the method of reattaching the drape unit according to some embodiments;

FIG. 11B is a schematic diagram for exemplifying the method of reattaching the drape unit according to some embodiments;

FIG. 12A is a perspective view for illustrating operations from reattaching the drape unit with cover to attaching the surgical instrument according to some embodiments;

FIG. 12B is a perspective view for illustrating operations from reattaching the drape unit with cover to attaching the surgical instrument according to some embodiments;

FIG. 13 is a perspective view illustrating the locking cover according to some embodiments;

FIG. 14 is a plan view illustrating the locking cover according to some embodiments;

FIG. 15 is a bottom view illustrating the locking cover according to some embodiments;

FIG. 16 is a right side view illustrating the locking cover according to some embodiments; and

FIG. 17 is a front view illustrating the locking cover according to some embodiments.

### DETAILED DESCRIPTION

A portion of a medical robot to which a surgical instrument is attached is provided with a movable member that transmits driving force to the surgical instrument and an opening that guides the movement of the movable member. If such an opening is provided between the clean area and the unclean area, the isolation between the clean area and the unclean area will be insufficient.

Hereinafter, one or more embodiments of the present disclosure will be described with reference to the drawings. In the following description, the same members are denoted by the same reference numerals and the description of members once explained may be omitted.

Various embodiments provide a draft unit that is interposed between a surgical instrument and a medical robot to sufficiently isolate the clean area and the unclean area from each other. Additionally, various embodiments provide a cover for protecting the drape unit when the surgical instrument is not attached. The cover allows for realignment of the drape unit such that the surgical instrument can be quickly and properly reattached to the drape unit.

### Configuration of Medical Robot and Drape Unit

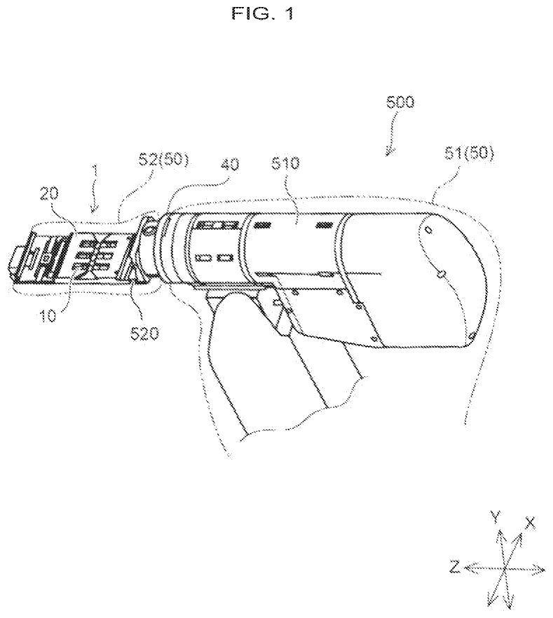

The following describes various embodiments with reference to FIGS. 1 to 4.

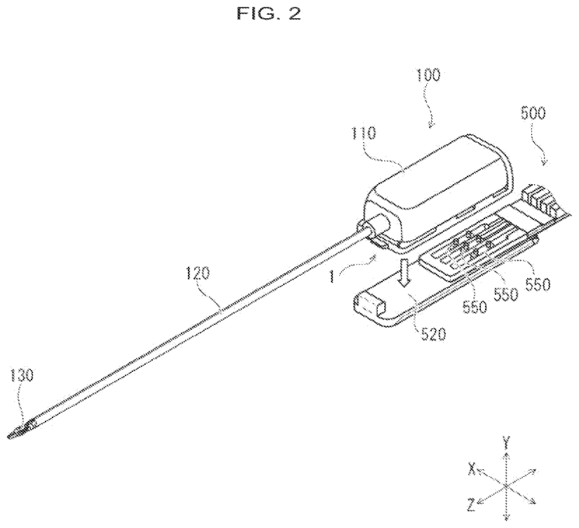

As illustrated in FIGS. 1 and 2, a medical robot 500 has a multi-degree-of-freedom arm 510 that is a manipulator capable of being remotely controlled. The tip portion of the multi-degree-of-freedom arm 510 is provided with an attaching portion 520 to which a drape unit 1 according to the present embodiment is attached. A surgical instrument 100 is attached to the multi-degree-of-freedom arm 510 via the drape unit 1.

The drape unit 1 is provided with a separator main body 20. The separator main body 20 is detachably attached to the attaching portion 520 of the multi-degree-of-freedom arm 510, and the surgical instrument 100 is detachably attached to the separator main body 20 of the drape unit 1. The drape unit 1 is provided with a drape 50 that covers outside of the medical robot 500. The drape unit 1 is disposed between the medical robot 500, which holds the surgical instrument 100, and the surgical instrument 100, and the drape unit 1 serves to isolate the surgical instrument 100 and the medical robot 500 from each other. The drape unit 1 also serves to transmit power in the forward/backward direction from power transmission parts 550 of the medical robot 500 to respective movable parts 150 provided in the surgical instrument 100.

For ease of description, the direction (forward/backward direction) of the power transmitted from the power transmission parts 550 to the drape unit 1 will be referred to as a Z direction, one of the directions orthogonal to the Z direction will be referred to as an X direction, and the direction orthogonal to the Z direction and the X direction will be referred to as a Y direction.

In some embodiments, the Z direction is also the direction in which the tip portion of the multi-degree-of-freedom arm 510 extends. The tip portion (attaching portion 520) of the multi-degree-of-freedom arm 510 is configured to rotate about an axis in the Z direction. An arm portion of the multi-degree-of-freedom arm 510 is also axially rotatable. The surgical instrument 100 attached to the medical robot 500 can therefore approach a patient from various angles using the multi-degree-of-freedom arm 510.

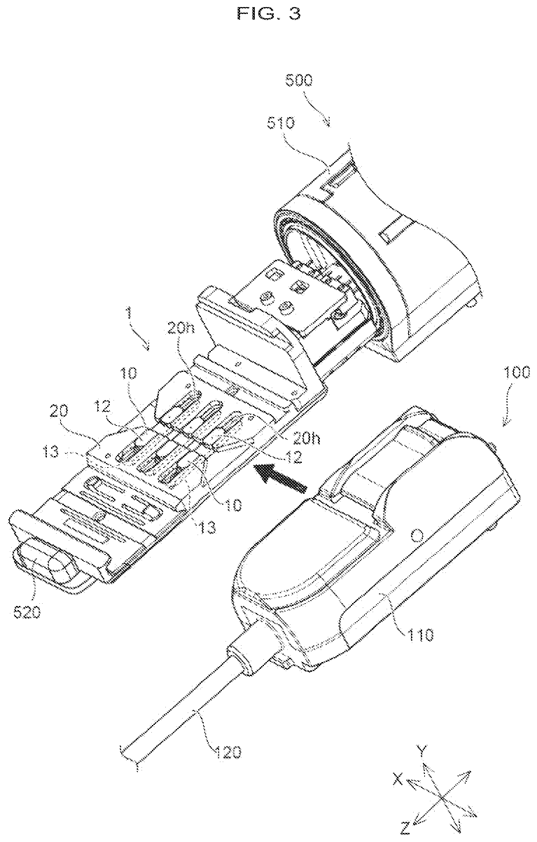

As illustrated in FIG. 2, the surgical instrument 100 comprises a main body 110, a shaft 120 extending from the main body 110, and a treatment part 130 provided at the tip of the shaft 120 (an end portion opposite to the main body 110). The treatment part 130 may be, for example, forceps. For easy understanding of the relationships between the surgical instrument 100 and drape unit 1 and the attaching portion 520 of the medical robot 500, FIG. 2 illustrates a situation in which the drape unit 1 in a state of being attached to the surgical instrument 100 is attached to the attaching portion 520 of the medical robot 500. As illustrated in FIG. 3, the drape unit 1 is first attached to the attaching portion 520 of the medical robot 500, and the surgical instrument 100 is then attached to the drape unit 1 which has been attached to the attaching portion 520.

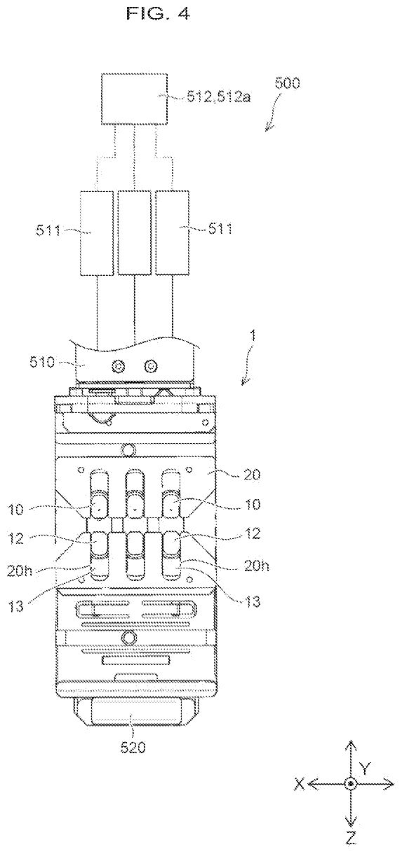

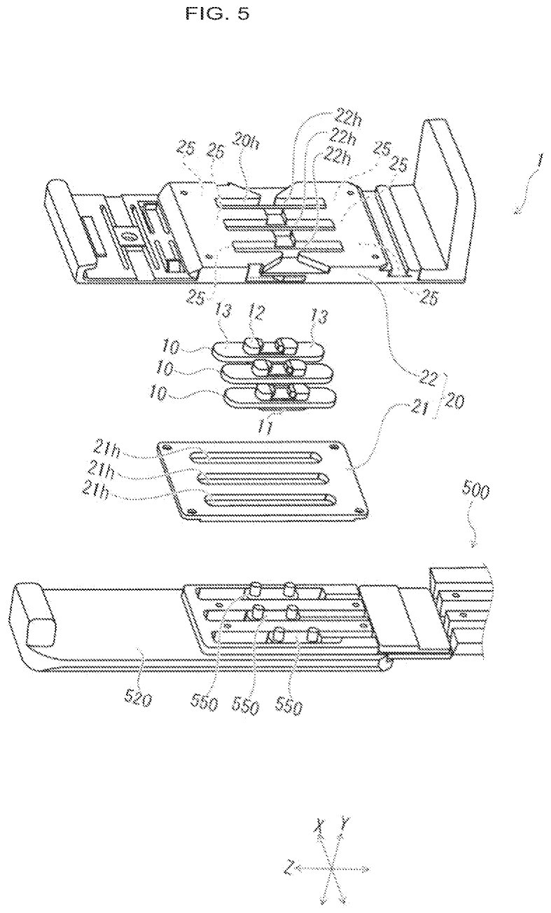

As illustrated in FIG. 4, the medical robot 500 is provided with actuator parts 511 and a control part 512. The actuator parts 511 generate driving force for operating the surgical instrument 100. The actuator parts 511 are connected to respective power transmission parts 550 (see FIG. 5) that transmit driving force in the forward/backward direction (Z direction) to the drape unit 1.

In some embodiments, the actuator parts 511 may generate driving force using a gas such as air or a fluid. The actuator parts 511 may use electric motors, and the type of generating the power is not limited. The actuator parts 511 may have a configuration using a piston and a cylinder or a configuration in which the driving force is generated from other known fluids, and the specific configuration is not limited.

The control part 512 controls generation of the driving force in the actuator parts 511. The control part 512 also controls the movement in the Z direction in the power transmission parts 550 and the arrangement positions of the power transmission parts 550. For easy understanding, an example in which the control part 512 controls the supply of a gas such as air to the actuator parts 511 will be described. The control part 512 may serve as an attachment control part 512a, which will be described later.

The main body 110 of the surgical instrument 100 is fitted into the separator main body 20 of the drape unit 1 to attach the surgical instrument 100 to the multi-degree-of-freedom arm 510, which has been attached to the attaching portion 520 of the multi-degree-of-freedom arm 510, so as to be slid, for example, in the X direction.

The power from the power transmission parts 550 can be transmitted to the main body 110 via the drape unit 1 due to the fitting of the main body 110 into the separator main body 20. Link means (e.g., wires) that transmit power to the treatment part 130 are provided in the main body 110, and the movement of the power transmission parts 550 can be transmitted from the drape unit 1 to the link means of the surgical instrument 100 to operate the treatment part 130.

The surgical instrument 100 to be attached to the medical robot 500 is sterilized and placed in a clean area. On the other hand, the medical robot 500 is placed in an unclean area, which is not sterilized to a same degree as the clean area. The drape unit 1, which separates the clean area and the unclean area, is also sterilized.

As illustrated in FIG. 1, the drape unit 1 includes a membrane-like drape 50. The drape 50 has a first drape 51 that covers the multi-degree-of-freedom arm 510 side from a joint member 40 having a bearing structure, and a second drape 52 that covers the attaching portion 520 side from the joint member 40. The first drape 51 is covered outside of the multi-degree-of-freedom arm 510, and is connected to cover the multi-degree-of-freedom arm 510 by the joint member 40. The second drape 52 is attached so that the separator main body 20 is attached to the attaching portion 520 in a state of being covered with the first drape 51 thereby to cover the attaching portion 520 side of the joint member 40.

### Detailed Structure of Drape Unit

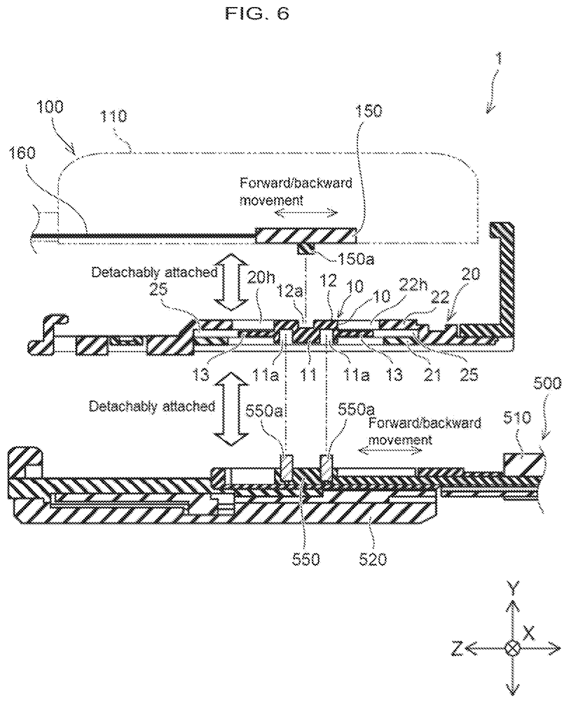

The following describes various embodiments with reference to FIGS. 5 and 6.

FIG. 6 illustrates a YZ cross section of the drape unit 1. For descriptive purposes, FIG. 6 illustrates the cross-sectional view of the drape unit 1, the cross-sectional view of the power transmission part 550 of the medical robot 500 and the cross-sectional view of the movable part 150 of the surgical instrument 100 in a state of being isolated from the drape unit 1.

As illustrated in FIGS. 5 and 6, the drape unit 1 includes sliders 10 that receive the power from the power transmission parts 550 of the medical robot 500 and a separator main body 20 that is detachably attached to the medical robot 500. The separator main body 20 has through-holes 20h through which respective parts of the sliders 10 are inserted.

The separator main body 20 is an assembly that comprises a first fixed portion 21 and a second fixed portion 22. The sliders 10 are arranged so as to be interposed between the first fixed portion 21 and the second fixed portion 22 and are housed so as not to drop off from the separator main body 20.

The separator main body 20 has housing portions 25 that cover respective parts of the sliders 10 as viewed in the penetrating direction (Y direction) of the through-holes 20h when the sliders 10 move to end portions (movable range ends) in the forward/backward direction. That is, the housing portions 25 of the separator main body 20 are configured to house respective parts of the sliders 10 when the sliders 10 move to the movable range ends in the Z direction, and the exposure of the sliders 10 to the outside is therefore suppressed. When the surgical instrument 100 is replaced during surgery, the separator main body 20 of the drape unit 1 is in a state of being exposed from when the surgical instrument 100 attached to the medical robot 500 is detached to when another surgical instrument 100 is attached. At this time, there is a risk that the practitioner or an assistant (supporting staff) may carelessly touch the separator main body 20 or some entity (e.g., a robot arm disposed next to the separator main body 20) may collide with the separator main body 20. In the medical robot 500 according to some embodiments, as described above, the movable range ends of the sliders 10 of the drape unit 1 in the Z direction are housed in the housing portions 25 of the separator main body 20, and therefore reducing the likelihood of problems, such as unexpected movement, dropping, and damage of the sliders 10 due to contact and/or collision.

Moreover, in the case of the drape unit 1 provided on the medical robot 500 as in some embodiments, the part of the medical robot 500 containing the attaching portion 520 and the actuator parts 511 provided with the power transmission parts 550 freely rotates and moves, and the through-holes 20h of the drape unit 1 face various directions accordingly. In the drape unit 1 according to some embodiments, the sliders 10 are housed in the separator main body 20 so as not freely move or fall out, and therefore even when the through-holes 20h of the drape unit 1 face any direction, the sliders 10 can appropriately prevent the exposure of the unclean area.

### Detailed Structure of Sliders

Each slider 10 has a first movable portion 11 that receives power from the corresponding power transmission part 550, a second movable portion 12 that transmits power to the corresponding movable part 150 of the surgical instrument 100, and a third movable portion 13 that extends from the second movable portion 12 in the forward/backward direction (Z direction).

The first movable portion 11 is provided with first engagement portions 11a into which protruding portions 550a of the power transmission part 550 are fitted when the drape unit 1 is attached to the attaching portion 520 of the multi-degree-of-freedom arm 510, allowing the power when the power transmission part 550 moves forward/backward to be transmitted to the first movable portion 11, and the slider 10 can move forward/backward.

The second movable portion 12 is provided with a second engagement portion 12a into which a protruding portion 150a, also referred to as movable part's protruding portion 150a, of the movable part 150 of the surgical instrument 100 is fitted. When the main body 110 of the surgical instrument 100 is attached to the drape unit 1, the movable part's protruding portion 150a protruding from the back surface of the main body 110 is fitted into the second engagement portion 12a of the second movable portion 12, allowing the slider 10 and the movable part 150 of the surgical instrument 100 to be engaged with each other, and the forward/backward movement of the power transmission part 550 can be transmitted from the slider 10 to the movable part 150 of the surgical instrument 100. That is, when the power transmission part 550 is moved forward/backward, the power is transmitted from the slider 10 to the movable part 150, and the forward/backward movement of the movable part 150 can be transmitted to a link means 160, for example, the wire 160, to operate the treatment part 130 via the link means 160.

The third movable portion 13 is a portion that extends from the second movable portion 12 in the Z direction (forward/backward direction). When viewed in the Y direction, the third movable portion 13 is provided larger than the second movable portion 12 and therefore serves to cover the corresponding through-hole 20h.

### Detailed Structure of Separator Main Body

The first fixed portion 21 of the separator main body 20 is provided with first openings 21h through which the first movable portions 11 of the sliders 10 are inserted, and the second fixed portion 22 is provided with second openings 22h through which the second movable portions 12 of the sliders 10 are inserted. When viewed in the Y direction, the areas in which the first openings 21h and the second openings 22h overlap are the through-holes 20h. The first openings 21h and the second openings 22h are elongated holes extending in the Z direction and guide the sliders 10 to move forward/backward in the Z direction within the range of the elongated holes.

That is, the sliders 10 can move forward/backward in the Z direction within the range of the elongated holes of the first openings 21h and the second openings 22h. In the movable regions of the sliders 10 between the first fixed portion 21 and the second fixed portion 22, portions that overlap the first fixed portion 21 as viewed in the Y direction are the housing portions 25.

### Drape Unit with Cover

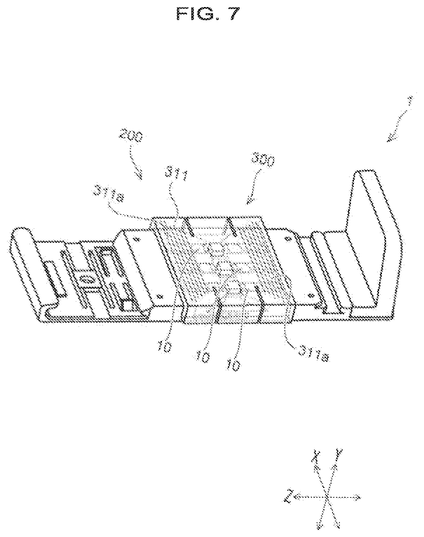

The following describes various embodiments with reference to FIGS. 7 to 9B.

FIG. 7 is a perspective view for exemplifying a drape unit with cover according to some embodiments.

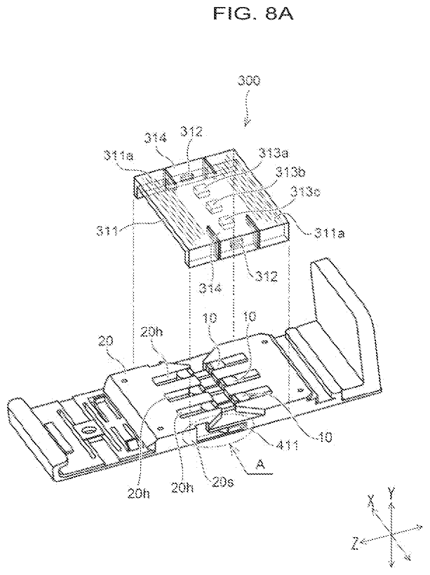

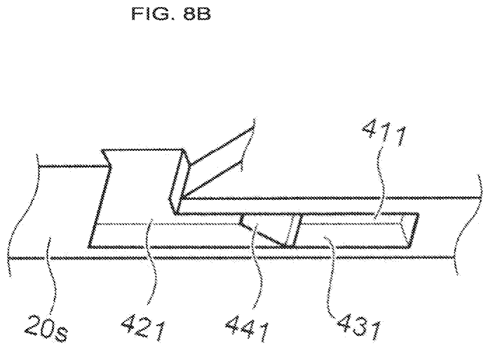

FIG. 8A is an exploded perspective view for exemplifying the drape unit with cover according to some embodiments, and FIG. 8B is an enlarged view of part A of FIG. 8A.

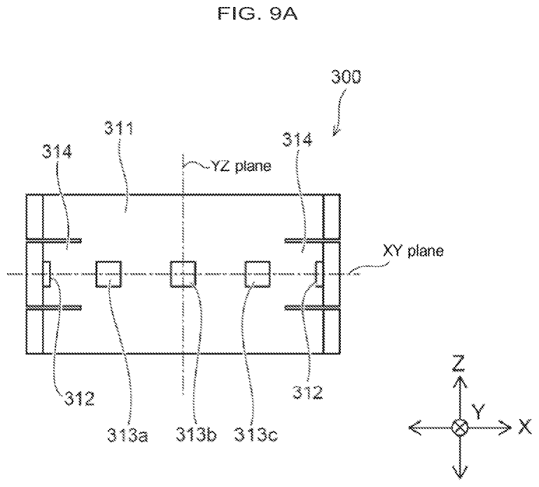

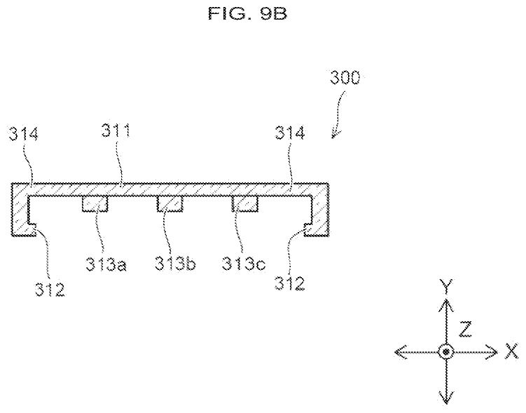

FIGS. 9A and 9B are diagrams for exemplifying a locking cover according to some embodiments. FIG. 9A illustrates a plan view of a locking cover 300 as viewed from the back surface, and FIG. 9B illustrates a cross-sectional view of the locking cover.

A drape unit with cover 200 includes the previously described drape unit 1 and a locking cover 300 that can be attached to and removed from the drape unit 1. The drape unit with cover 200 is applied when the drape unit 1 is attached to or reattached to the attaching portion 520 of the medical robot 500.

In the drape unit with cover 200 in which the locking cover 300 is attached to the drape unit 1, the locking cover 300 covers the entire through-holes 20h provided in the separator main body 20, and the isolation between the clean area and the unclean area can therefore be ensured.

When the drape unit 1 is attached to the medical robot 500, the drape 50 may be jammed by mistake, or the drape unit 1 may not be attached at the correct position, resulting in incorrect attachment. In such a case, the drape unit 1 may be detached from the attaching portion 520 and attached again (reattached) after dealing with the jamming or reconsidering the attaching position.

When the drape unit 1 is reattached, the relative positions of the sliders 10 with respect to the separator main body 20 are maintained by attaching the locking cover 300 to the drape unit 1, and the drape unit 1 can be attached accurately in a short time.

### Locking Cover

The locking cover 300 includes a main body portion 311, holding portions 314, and slider holding portions 313a, 313b, and 313c. When the slider holding portions 313a, 313b, and 313c are collectively referred to without distinction, they will be referred to as slider holding portions 313.

The main body portion 311 is a plate-shaped member that can cover the entire through-holes 20h when the locking cover 300 is attached to the separator main body 20 of the drape unit 1. The surface of the main body portion 311 may be provided with protrusions 311a. By providing the protrusions 311a, when the locking cover 300 is slid on the separator main body 20, the locking cover 300 is easily caught on a finger, and the sliding operation is facilitated. In some embodiments, a height of the protrusions 311a is 0.1 mm or more and 0.5 mm or less, and a more specific example is 0.25 mm or more and 0.35 mm or less.

The holding portions 314 are vertical pieces provided at both end portions of the main body portion 311 in the width direction, and are portions that detachably hold the relative position of the main body portion 311 with respect to the separator main body 20. The holding portions 314 engage with the separator main body 20 when the locking cover 300 is attached to the separator main body 20 so as to embrace both side surfaces 20s of the separator main body 20.

In some embodiments, the holding portions 314 are provided in a state of having spring properties relative to the main body portion 311, and convex portions 312 protruding inward are provided at positions of the holding portions 314 facing the side surfaces 20s of the separator main body 20. Both the side surfaces 20s of the separator main body 20 of the drape unit 1 are provided with locking reception portions 411 that receive the convex portions 312 to restrict movement of the convex portions 312 in the forward/backward direction (Z direction).

The slider holding portions 313 engage with the second engagement portions 12a of the sliders 10 when the locking cover 300 is attached to the separator main body 20. In the present embodiment, the slider holding portions 313a, 313b, and 313c engage with respective second engagement portions 12a of three sliders 10. By engaging the slider holding portions 313 with the second engagement portions 12a, the relative positions between the main body portion 311 and the sliders 10 are maintained.

Therefore, when the locking cover 300 is placed over the separator main body 20 and the convex portions 312 of the holding portions 314 are fitted into the locking reception portions 411 to hold the locking cover 300, the slider holding portions 313 maintain the relative positions of the sliders 10, which allows the sliders 10 to be accurately engaged with the protruding portions 550a of the power transmission parts 550 in the first engagement portions 11a.

Each side surface 20s of the separator main body 20 is provided with an initial reception portion 421 and a guide portion 431. The initial reception portion 421 receives the convex portion 312 provided on the holding portion 314 to temporarily hold the locking cover 300. The guide portion 431 guides the convex portion 312 in the forward/backward direction from the initial reception portion 421 to the locking reception portion 411. In addition, the locking reception portion 411 is provided with a locking wall 441.

The initial reception portion 421, guide portion 431, and locking reception portion 411 are formed, for example, by grooves provided in the side surface 20s of the separator main body 20. The grooves extend in the forward/backward direction (Z direction), and the initial reception portion 421 is arranged on one side in the forward/backward direction while the locking reception portion 411 is arranged on the other side. The locking wall 441 is provided on the initial reception portion 421 side of the locking reception portion 411.

When the convex portion 312 is engaged with the initial reception portion 421 to temporarily hold the locking cover 300, each slider holding portion 313 can engage with the second engagement portion 12a of the corresponding slider 10 which has moved to an end on one side of the through-hole 20h in the forward/backward direction.

The locking wall 441 has an inclined surface that becomes higher in the direction from the initial reception portion 421 toward the locking reception portion 411 (away from the side surface 20s in the X direction). Such a locking wall 441 allows the convex portion 312 to be guided to the guide portion 431 and move in one of the forward and backward directions (direction from the initial reception portion 421 toward the locking reception portion 411), but can restrict the movement of the convex portion 312 in the other direction (the direction from the locking reception portion 411 toward the initial reception portion 421).

The main body portion 311 of the locking cover 300 may have optical transparency. The optical transparency of the main body portion 311 makes it easier to visually recognize the relative positions of the sliders 10 with respect to the separator main body 20 in a state in which the locking cover 300 is held by the separator main body 20.

As illustrated in FIG. 9A, the locking cover 300 may have a symmetrical shape with a predetermined cross section as a plane of symmetry. This plane of symmetry may be a plane (YZ plane) including a direction along the forward/backward direction (Z direction) or a plane (XY plane) orthogonal to the forward/backward direction (Z direction). The symmetry of the locking cover 300 allows the locking cover 300 to be attached to the drape unit 1 regardless of the orientation in the forward/backward direction.

### Method of Reattaching Drape Unit

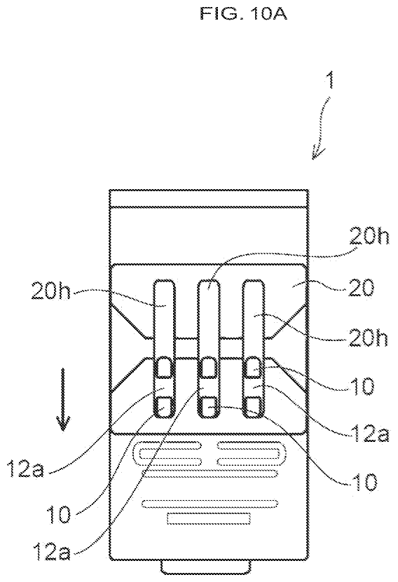

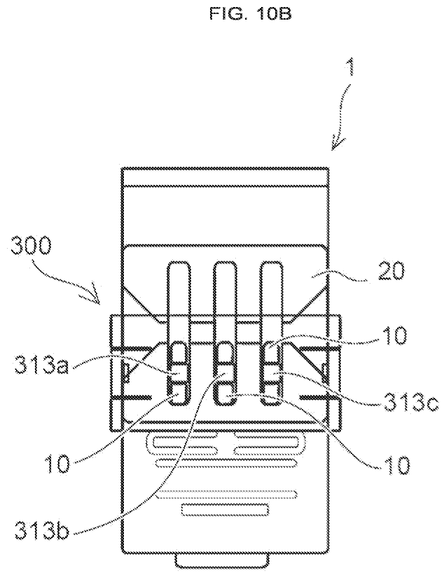

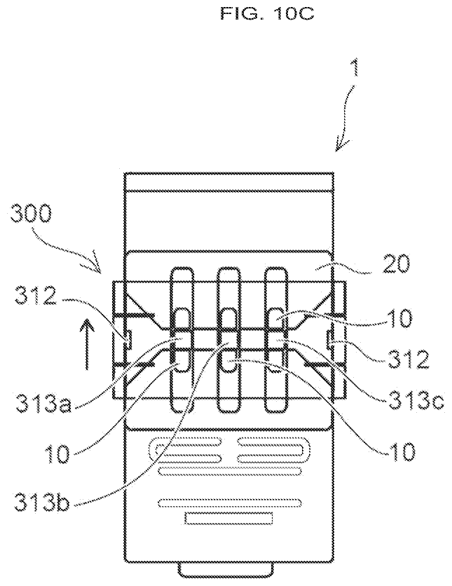

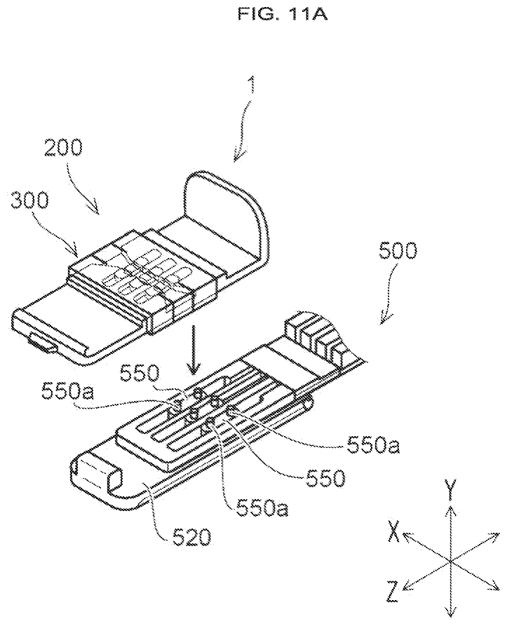

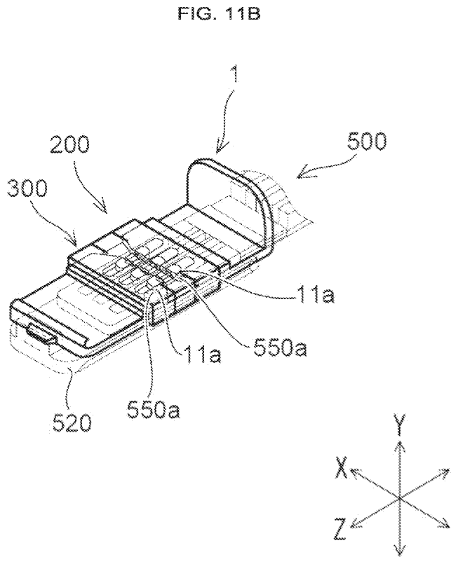

FIGS. 10A to 11B are schematic diagrams for exemplifying a method of reattaching the drape unit according to some embodiments.

As illustrated in FIG. 10A, in a state in which the drape unit 1 is detached from the attaching portion 520, the sliders 10 are moved to an end on one side of the separator main body 20. In the state in which the drape unit 1 is detached from the attaching portion 520, the sliders 10 and the power transmission parts 550 are not engaged and the sliders 10 are free to move. Therefore, by turning the distal end side of the separator main body 20 downward, for example, the sliders 10 move downward by their own weights and move to the lowest positions along the through-holes 20h.

As illustrated in FIG. 10B, the locking cover 300 is attached to the separator main body 20. In this attaching, the convex portions 312 provided on the holding portions 314 of the locking cover 300 are engaged with the initial reception portions 421 to temporarily hold the locking cover 300. In the state in which the locking cover 300 is temporarily held, the slider holding portions 313 of the locking cover 300 are engaged with the second engagement portions 12a of the sliders 10, which have moved to the end on one side of the separator main body 20, and the sliders 10 can be held in a state of being positioned at the end on one side of the separator main body 20.

As illustrated in FIG. 10C, the position of the locking cover 300 relative to the separator main body 20 is slid to the other side in the forward/backward direction. In this operation, when the protrusions 311a (see FIGS. 7 and 8A) are provided on the surface of the main body portion 311 of the locking cover 300, the locking cover 300 is easily caught on a finger, and the sliding operation is facilitated. By this sliding operation, the convex portions 312 of the holding portions 314 are guided from the initial reception portions 421 to the guide portions 431 over the locking walls 441 and reach the locking reception portions 411, which allows the relative position of the locking cover 300 with respect to the separator main body 20 to be fixed. In this operation, as the locking cover 300 slides, the sliders 10 engaging with the slider holding portions 313 also move. In some embodiments, when the convex portions 312 are received in the locking reception portions 411 and the locking cover 300 is fixed at a predetermined position, the sliders 10 are placed at neutral positions in the forward/backward direction through the through-holes 20h, for example, centered in the through-holes 20h.

As illustrated in FIG. 11A, the drape unit with cover 200 to which the locking cover 300 is fixed at the predetermined position is reattached to the attaching portion 520 of the medical robot 500. In this operation, the actuator parts 511 which drive the power transmission parts 550 are controlled by the control part 512 so that the power transmission parts 550 are held at predetermined positions in the forward/backward direction. In some embodiments, for example, the control part 512 illustrated in FIG. 4 is made to serve as the attachment control part 512a, and the actuator parts 511 are controlled to move the power transmission parts 550 to predetermined positions (e.g., neutral positions) in the forward/backward direction. When the drape unit with cover 200 is attached to the attaching portion 520 in this state, the protruding portions 550a of the power transmission parts 550 can be accurately engaged with the first engagement portions 11a of the sliders 10 of the drape unit 1 (see FIG. 11B).

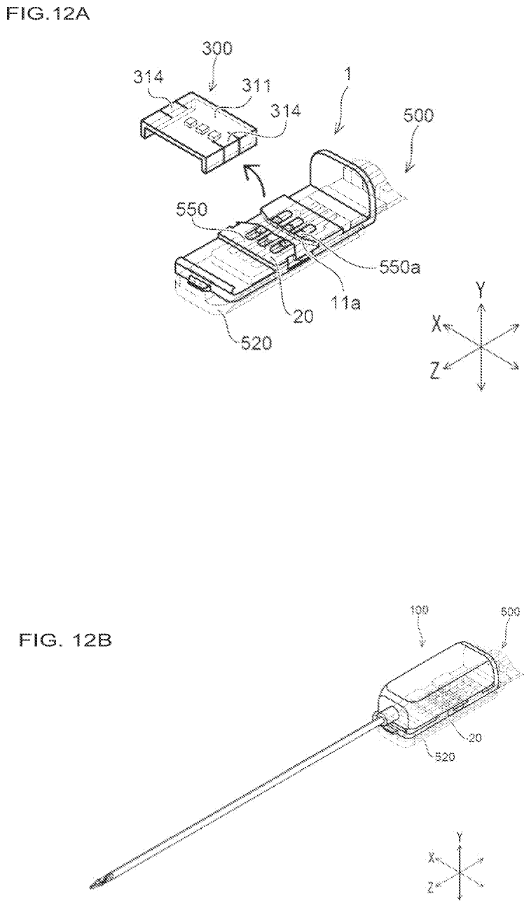

FIGS. 12A and 12B are perspective views for exemplifying steps from reattaching the drape unit with cover to attaching the surgical instrument according to some embodiments.

As illustrated in FIG. 12A, after the drape unit with cover 200 is reattached to the attaching portion 520 of the medical robot 500, the locking cover 300 is detached. The position of the slider 10 does not shift after the locking cover 300 is detached because the first engagement portions 11a of the sliders 10 and the protruding portions 550a of the power transmission parts 550 are engaged. In some embodiments, the locking cover 300 is designed to be easily detached along the XY plane. In some embodiments, the holding portions 314 are provided in a cantilever shape with cuts in the X direction relative to the main body portion 311. Therefore, by hooking one holding portion 314 with the thumb, for example, and pulling it up along the XY plane, the one holding portion 314 can be detached so as to rotate about the other holding portion 314 as a fulcrum, allowing the locking cover 300 to be easily detached with one hand.

As illustrated in FIG. 12B, the surgical instrument 100 is attached to the separator main body 20 of the drape unit 1. In this operation, since all the sliders 10 of the separator main body 20 are arranged at the neutral positions, the second engagement portions 12a of the sliders 10 are aligned in the X direction, allowing the surgical instrument 100 to be attached to an accurate position by sliding the main body 110 of the surgical instrument 100 in the X direction and fitting it in (see FIG. 3).

Thus, when the drape unit 1 is reattached, the locking cover 300 is attached to the drape unit 1, which has been once detached from the attaching portion 520, and is temporarily held, and the locking cover 300 is slid thereby to allow all the sliders 10 to be aligned at predetermined positions (e.g., neutral positions) with respect to the separator main body 20, allowing the positions of the sliders 10, which are free because the drape unit 1 is detached, to be maintained and aligned at the positions required for reattaching, and when the drape unit with cover 200 is reattached to the attaching portion 520, the first engagement portions 11a of the sliders 10 and the protruding portions 550a of the power transmission parts 550 can be engaged accurately and in a short time. It is therefore possible to reattach the drape unit 1 while maintaining hygiene without touching the drape unit 1 unnecessarily.

### Another Example of Locking Cover

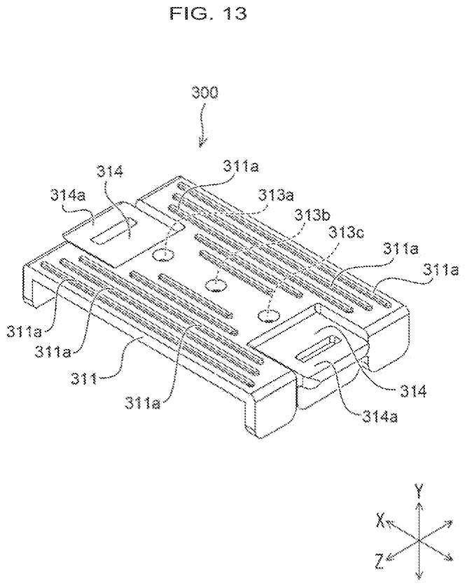

FIG. 13 is a perspective view illustrating the locking cover according to some embodiments.

FIG. 14 is a plan view illustrating the locking cover according to some embodiments.

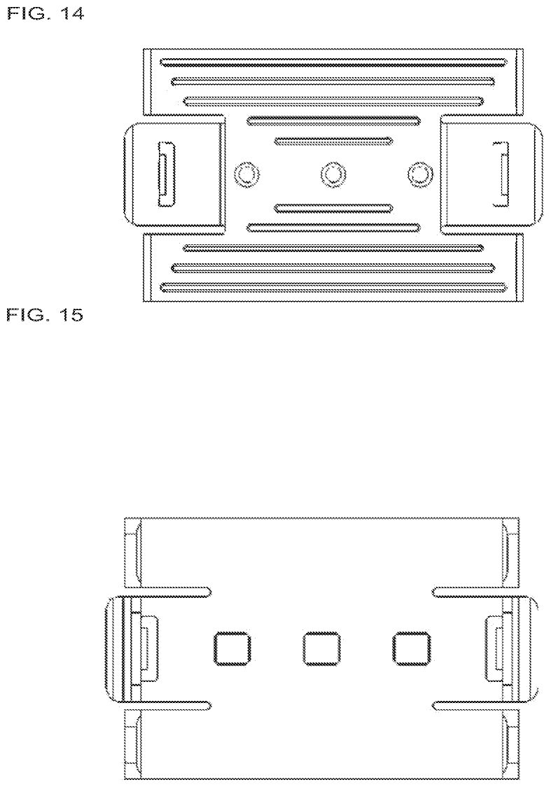

FIG. 15 is a bottom view illustrating the locking cover according to some embodiments.

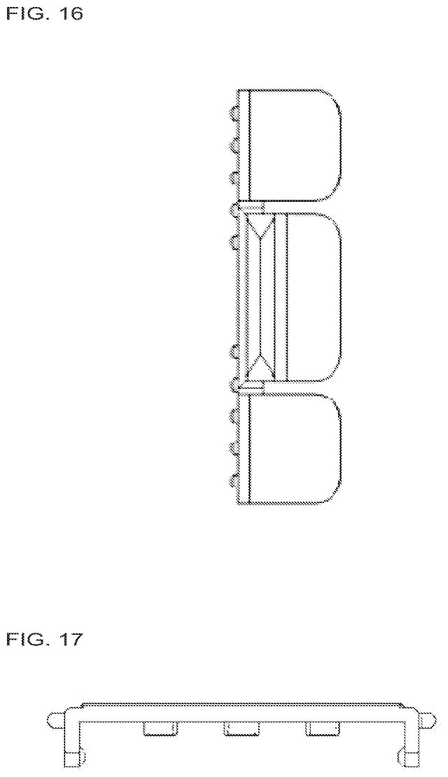

FIG. 16 is a right side view illustrating the locking cover according to some embodiments.

FIG. 17 is a front view illustrating the locking cover according to some embodiments.

In some embodiments, the left side view of the locking cover 300 is symmetrical with the right side view, and the rear view is symmetrical with the front view.

In some embodiments, the holding portions 314 of the locking cover 300 provided at both end portions of the main body portion 311 in the width direction are provided with extending portions 314a. The extending portions 314a are provided so as to extend from the corner portions of the holding portions 314 in the X direction. When the holding portions 314 are provided at both end portions of the main body portion 311 in the width direction, the extending portions 314a extending outward in the X direction from the corner portions of respective holding portions 314 are provided.

The extending length of the extending portions 314a may be a length that allows fingers to be easily caught on the extending portions 314a. In some embodiments, the extending length of the extending portions 314a may be 1 mm or more and 5 mm or less. In some embodiments, the extending length may be 2 mm or more and 3 mm or less. Each extending portion 314a may be provided over the entire area of the holding portion 314 in the Z direction, or may also be provided in a part of the holding portion 314 in the Z direction.

The extending portions 314a results in an effect as follows when the locking cover 300 attached to the attaching portion 520 of the medical robot 500 is detached. That is, when the extending portion 314a of one of the holding portions 314 is hooked, for example, with a thumb to pull it up along the XY plane, the locking cover 300 can be easily detached by rotating it using the other holding portion 314 as a fulcrum.

As described above, according to some embodiments, it is possible to provide the drape unit with cover 200, the locking cover 300, and the medical robot 500 with which the clean area and the unclean area can be sufficiently isolated from each other, and when the drape unit interposed between the medical robot and the surgical instrument is reattached, the attachment can be completed accurately and in a short time.

Although the various embodiments have been described above, the present disclosure is not limited thereto. For example, in the above embodiments, three sliders 10 are arranged in parallel, but the number of the sliders 10 is not limited. In the above embodiments, the treatment part 130 of the surgical instrument 100 is described as forceps, but a treatment part 130 other than the forceps may be employed.

While various embodiments have been described, the present disclosure is not limited to the above embodiments and the various embodiments may be improved or modified within the scope of the present disclosure.

Thus, it should be understood that the present disclosure is not limited to the above embodiments, but various other changes and modifications may be made therein without departing from the spirit and scope of the appended claims.

1. A drape unit, comprising:
a drape unit disposed between a medical robot that holds a surgical instrument and the surgical instrument to isolate the surgical instrument and the medical robot from each other, the drape unit comprising:
a slider having a first engagement portion engaged with a power transmission part of the medical robot configured to transmit power in a forward/backward direction and a second engagement portion engaged with a movable part of the surgical instrument, the slider transmitting power received from the power transmission part to the movable part; and
a separator main body having a through-hole through which at least a part of the slider is inserted, the separator main body being detachably attached to the medical robot; and
a locking cover configured to be detached and reattached to the drape unit, the locking cover comprising:
a main body portion configured to cover an entirety of the through-hole;
a holding portion that detachably holds a relative position of the main body portion to the separator main body; and
a slider holding portion provided on the main body portion so as to hold a relative position between the main body portion and the slider, the slider holding portion being configured to engage with the second engagement portion.

2. The drape unit according to claim 1, wherein the main body portion has optical transparency, and a relative position of the slider with respect to the separator main body can be visually recognized in a state in which the locking cover is attached to the separator main body.

3. The drape unit according to claim 1, wherein
the holding portion comprises a convex portion that protrudes toward a side of the separator main body,
the drape unit further includes a locking reception portion configured to receive the convex portion and restrict movement of the convex portion in the forward/backward direction, and
when the locking cover is attached to the separator main body by the locking reception portion, the slider, whose relative position is held by the slider holding portion, can be engaged with the power transmission part.

4. The drape unit according to claim 3, wherein
the drape unit further includes:
an initial reception portion configured to receive the convex portion to temporarily hold the locking cover; and
a guide portion configured to guide the convex portion in the forward/backward direction from the initial reception portion to the locking reception portion, and
the locking reception portion is provided with a locking wall configured to allow the convex portion to be guided by the guide portion and move in one of forward and backward directions but restrict the movement of the convex portion in the other of the forward and backward directions.

5. The drape unit according to claim 4, wherein in a state in which the locking cover is temporarily held by the initial reception portion, the one or more slider holding portions are positioned to engage with the one or more second engagement portions of the slider at an end on one side of the through-hole in the forward/backward direction.

6. The drape unit according to claim 1, wherein the locking cover is a symmetrical shape with a predetermined cross section as a plane of symmetry.

7. The drape unit according to claim 6, wherein in a state in which the locking cover is attached to the drape unit, the plane of symmetry is a plane including a direction along the forward/backward direction of the drape unit.

8. The drape unit according to claim 6, wherein in a state in which the locking cover is attached to the drape unit, the plane of symmetry is a plane orthogonal to the forward/backward direction of the drape unit.

9. The drape unit according to claim 6, wherein the slider holding portion is equally divided by the plane of symmetry.

10. A locking cover for covering a drape unit comprising a slider having a first engagement portion engaged with a power transmission part of a medical robot configured to transmit power in a forward/backward direction and a second engagement portion engaged with a movable part of the surgical instrument and a separator main body having a through-hole through which at least a part of the slider is inserted 1, the locking cover comprising:
a main body portion configured to cover the entire through-hole;
a holding portion that detachably holds a relative position of the main body portion to the separator main body); and
a slider holding portion that is provided on the main body portion so as to hold a relative position between the main body portion and the slider and can be engaged with the second engagement portion.

11. An assembly comprising,
a medical robot comprising an arm and an attaching portion provided at an end of the arm, the attaching portion including a power transmission part and the arm including an attachment control part configured to control an actuator part that drives the one or more power transmission part so that the power transmission part is held at a predetermined position in a forward/backward direction:
a drape unit positioned between the medical robot and a surgical instrument configured to transmit power in a forward/backward direction from the medical robot,
the drape unit comprising:
one or more slider, each having a first engagement portion engaged with the power transmission part and a second engagement portion on an opposite side of the first engagement portion, the one or more slider transmitting the power received from the power transmission part; and
a separator main body having one or more through-holes through which a part of a respective one of the one or more sliders is inserted, the separator main body being detachably attached to the medical robot, and
a drape configured to isolate the surgical instrument and the medical robot from each other.

12. The assembly according to claim 11, further comprising,
a locking cover configured to be attached to the drape unit, the locking cover comprising:
a main body portion configured to cover an entirety of the through-hole;
a holding portion that detachably holds a relative position of the main body portion to the separator main body; and
a slider holding portion provided on the main body portion so as to hold a relative position between the main body portion and the slider, the slider holding portion being configured to engage with the second engagement portion.

13. The assembly according to claim 11, further comprising,
a surgical instrument configured to detachably attach to the drape unit, the surgical instrument comprising a moveable part.

14. The assembly according to claim 13, wherein the movable part comprises a protruding portion and the second engagement portion is configured to engage with the protruding portion so as to transmit the power from the power transmission part to the moveable part.

15. The assembly according to claim 11, wherein the drape comprises a first drape configured to covers the arm of the medical robot and a second drape configured to cover the attaching portion of the medical robot.

16. The assembly according to claim 11, wherein the separator main body comprises a first fixed portion and a second fixed portion and the one or more slider is interposed between the first fixed portion and the second fixed portion.

17. The assembly according to claim 15, wherein the one or more slider is configured to cover an entirety of a corresponding one of the one or more through-hole.
